(19) United States
(12) Patent Application Publication
(10) Pub. No.: US 2022/0280698 A1
(43) Pub. Date: Sep. 8, 2022

(54) POLYMER BASED HYDROGELS, AEROGELS, AND COMBINATIONS THEREOF

(71) Applicant: Georgia Tech Research Corporation, Atlanta, GA (US)

(72) Inventors: Cameron William Irvin, Acworth, GA (US); Meisha Shofner, Atlanta, GA (US)

(21) Appl. No.: 17/686,928

(22) Filed: Mar. 4, 2022

Related U.S. Application Data
(60) Provisional application No. 63/157,216, filed on Mar. 5, 2021.

(51) Int. Cl. A61L 27/52; A61L 27/48

(52) U.S. Cl. CPC A61L 27/52; A61L 27/48; A61L 2430/38

(57) ABSTRACT

Disclosed herein are hybrid materials including a polyvinyl alcohol component combined with a filler. The filler may be one or more of crystalline nanocellulose and chitin nanofibers. The hybrid materials may also include water, and in such cases are designated hydrogels. The hybrid materials may be substantially free of solvents including water, and in such cases are designated aerogels. The hydrogels, aerogels, and combinations thereof may be advantageously included in biomimetic implants, including as a biomimetic intervertebral disc replacement

### CROSS-REFERENCE TO RELATED APPLICATION

This application claims the benefit of U.S. Provisional Application 63/157,216, filed on Mar. 5, 2021, the contents of which are hereby incorporated in its entirety.

### FIELD OF THE INVENTION

This application is directed to hybrid materials including a polyvinyl alcohol component combined with a filler. The filler may be one or more of crystalline nanocellulose and chitin nanofibers. The hybrid materials may also include water, and in such cases are designated hydrogels. The hybrid materials may be substantially free of solvents including water, and in such cases are designated aerogels. The hydrogels, aerogels, and combinations thereof may be advantageously included in biomimetic implants, including as a biomimetic intervertebral disc replacement

### BACKGROUND

Spinal fusion is one of the most common surgical remedies for back pain despite concerns that it is an archaic procedure that limits range of motion in patients. Artificial intervertebral discs (IVD) are becoming a more popular replacement for spinal fusion due to their biomimicry, but the introduction of a dynamic implant without assured cellular adhesion runs the risk of becoming dislodged. Cellulose and chitin are promising materials that are finding their way into many biomedical applications. With great biocompatibility and excellent stiffness and crystallinity, these renewable materials offer the ability both to provide structural stability and enhanced cellular adhesion in implants. However, these fibers and crystals pose processing issues in difficulty of dispersion, relatively low stability temperature, and nanofiller aggregation.

In addition to generating useful fundamental polymer processing information for biomedical and other scientific applications, the successful integration of cellulose and chitin nanofibers and crystals into a three-part polymer composite of solids, hydrogels, and foams for a total intervertebral disc replacement will create a device capable of effectively handling physiological loads and adhering firmly to the surrounding tissue.

Back pain has been reported in 80% of the world's population with 75% of these cases being attributed to degenerated discs. Artificial intervertebral discs are a growing technology that seeks to replace the damaged fibrocartilage discs in an effort to supplant the more common spinal fusion. Cellulose and chitin are emerging materials that have displayed relatively low density, high aspect ratio, crystallinity, strength, and abundance. As biomaterials, cellulose and chitin have excellent biocompatibility, little to no toxicity, and they encourage cellular growth given their natural role in cellular systems. Therefore, their incorporation into polymers enhances strength and provides points of growth for cells. Cellulose and chitin are two of the most abundant resources on the planet and can be formed into crystals and fibers, but they face difficulties in processing, particularly in regard to dispersion. The goal is find an efficient way to process these fibers and crystals, implement them into a polymeric composite in various forms, and appropriately mimic the nature of collagen fibers in natural fibrocartilage.

The structure of intervertebral discs consists of two major components: (1) outer fibrous ring and (2) inner gel-like center. The fibrous ring consists of layered fibrocartilage, which is a composite of collagen fibers and cartilaginous tissue. The inner gel-like center is a soft structure containing loosely dispersed collagen fibers. One polymer that has been investigated for IVD replacement is poly(vinyl alcohol) (PVA). PVA is highly water-soluble and is used in various cartilage applications due to its low protein adsorption characteristics, biocompatibility, and chemical resistance. PVA can be generated in three forms (solids, hydrogels, foams) for use in a disc replacement. The exemplary material and device synergistically use the interactions between PVA and cellulose/chitin nanomaterials in provide, in some embodiments, a total unified disc replacement using all three forms.

Nanofillers are a type of material possessing at least one dimension under 100 nm, which can be incorporated into a larger matrix in an attempt to impart its properties on the overall structure. Their inclusion can improve the conductive properties, mechanical properties, and/or thermal stability, among many other properties of whatever matrix they are embedded within. Among the most commonly used nanofillers are carbon black, graphene, silica, and clay. A major driving factor for this shift to these nanomaterials is the mechanical properties that they offer. These nanomaterials allow for strengthening of polymers and, therefore, a customization of their mechanical properties for a variety of applications. However, there are some concerns about the environmental impact of some of these nanomaterials. Therefore, there has been a growing interest in renewable materials to take advantage of an abundant source for nanofillers and to improve ecological safety of the resulting high-performance materials.

Renewable materials are a class of materials that are derived from natural resources and have continued to garner popularity and development into a large field of study under materials science. In a sense, “nanocomposites” are common in nature, where materials such as nacre and bone are made up of multiscale structures that rely on ordered microstructures and interface interactions to impart strength and stiffness on a larger matrix. Scientists have tried to mimic these natural structures in part by utilizing nanomaterials derived from natural resources and repurposing them for the same reinforcement role in polymer matrices. These resulting nanocomposites are often called “green materials” and they have been working to replace non-renewable nanomaterials to improve sustainability and thermomechanical properties in multiple markets including the automotive and packaging industries, as well as improving biocompatibility in biomedical applications. “Green materials” also encompass polymeric materials aiming to replace petroleum-based materials, as oil availability is continually declining and its impact on the environment has received much scrutiny in the last few decades. These replacements for petroleum-based materials encompass biobased polymers that are biodegradable or compostable, thus reducing their overall negative impact on the world's ecosystem.

In the biomedical field, polymers and polymer composites have been a growing material of study in the use of implants, drug delivery, and tissue engineering. Titanium is one of the most commonly used materials for hard implants, but it is difficult to machine, is radiopaque, and possesses strength much greater than that of human bone/tissue. It is a common rule within biomedical device design that the utilized materials in implants should only be as strong as the material they are replacing (i.e. bone, cartilage, etc.), otherwise they may absorb too much of the load from the surrounding tissue, thus leading to early failure and potential damage to the patient. Polymers, such as poly(ether ether ketone) (PEEK), poly(lactic acid) (PLA), and PVA, have gained greater development into a wide variety of biomedical applications because they have a closeness in mechanical properties to natural human tissue in addition to ease of machinability and customizability, as well as radiolucency. Provided that renewable materials are extracted from biological sources, their introduction into biomedical products may also allow for an enhancement in biocompatibility and mechanical customizability without risk of inducing a negative response from the surrounding tissue. Patient specific devices have grown increasingly popular and the modular nature of renewable bioproduct polymer composites allows for the ability to tune the materials to the needs of the recipient.

There remains a need for improved polymer and polymer composites. There remains a need for improved polymer and polymer composites derived from renewable resources. There remains a need for improved biomedical implants with enhanced stability, biocompatibility, and mechanical tunability.

In accordance with the purposes of the disclosed materials and methods, as embodied and broadly described herein, the disclosed subject matter, in one aspect, relates to compounds, compositions and methods of making and using compounds and compositions.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

The details of one or more embodiments are set forth in the descriptions below. Other features, objects, and advantages will be apparent from the description and from the claims.

### BRIEF DESCRIPTION OF THE FIGURES

FIG. 1 depicts a schematic of the generation of different hybrid materials.

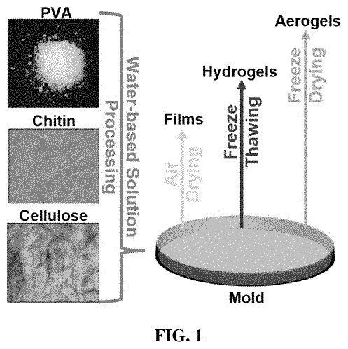

FIG. 2 depicts polarized optical microscopy images of (a) Neat HPVA, (b) 5CNC/HPVA, (c) 4CNC/1ChNF/HPVA, (d) 2.5CNC/2.5ChNF/HPVA, (e) 1CNC/4ChNF/HPVA, (f) 5ChNF/HPVA, (g) Neat LPVA, (h) 5CNC/LPVA, (i) 4CNC/1ChNF/LPVA, (j) 2.5CNC/2.5ChNF/LPVA, (k) 1CNC/4ChNF/LPVA, and (1) 5ChNF/LPVA.

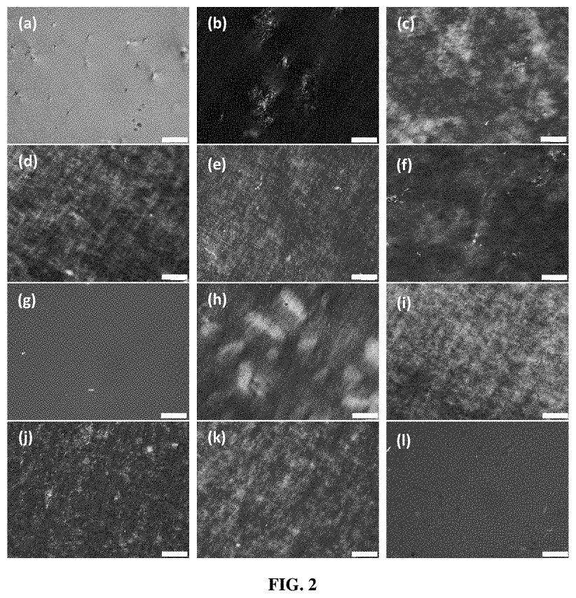

FIG. 3 depicts Zeta Potential of CNC and ChNF suspensions at various ratios.

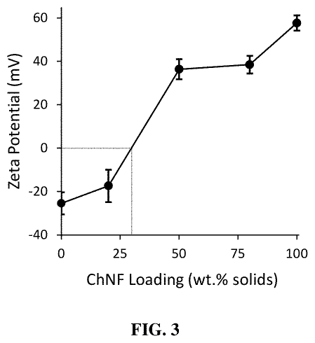

FIG. 4 depicts representative stress-strain curves of (a) HPVA-based samples and (b) LPVA-based samples.

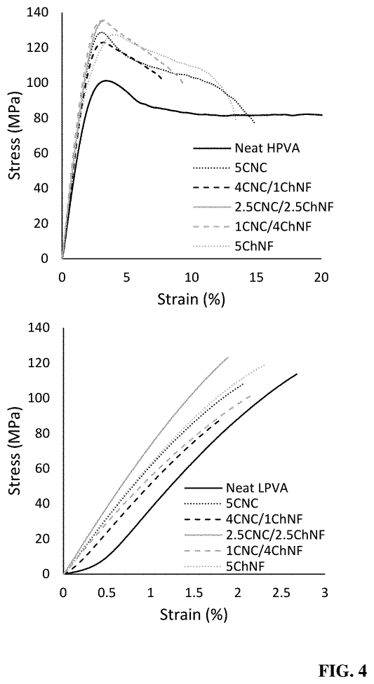

FIGS. 5A-5C depict water absorption change for (FIG. 5A) neat PVA and (FIG. 5B) 5ChNF over 360 minutes for 1, 3, 5, and 7FT cycles; (FIG. 5C) water absorption in neat PVA and nanocomposite hydrogels over 1, 3, 5, and 7 freeze-thaw cycles after six hours. * indicates statistical difference from the neat PVA hydrogel sample set.

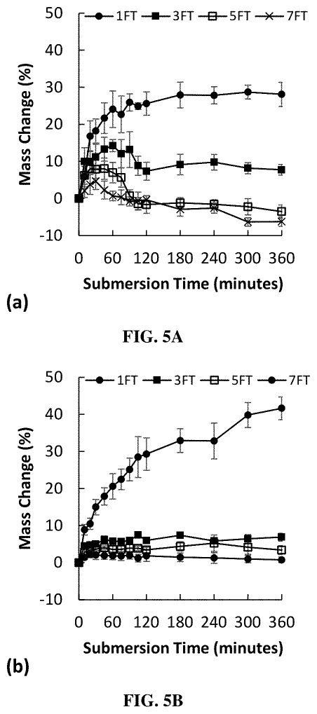

FIGS. 6A-6C depict representative curves for stress at 50% compressive strain for CNC- and ChNF-reinforced PVA hydrogel composites for (FIG. 6A) one, (FIG. 6B) three, and (FIG. 6C) seven FT cycle(s).

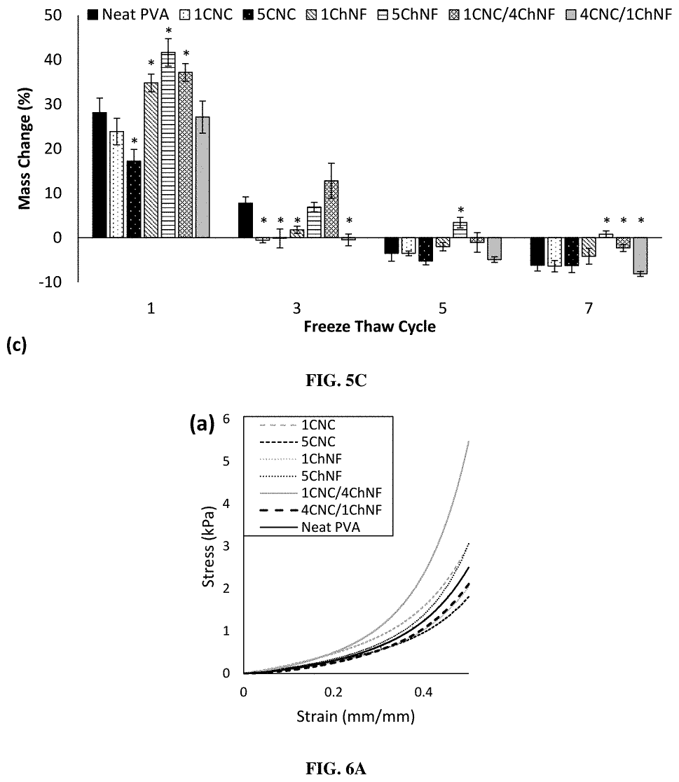

FIG. 7 depicts SEM images at 100× (left) and 1000× (right) magnification of cryo-fractured aerogel surfaces. (a, b) Neat PVA, (c, d) 1CNC, (e, f) 1ChNF, (g, h) 0.2CNC/0.8ChNF, and (i, j) 0.8CNC/0.2ChNF.

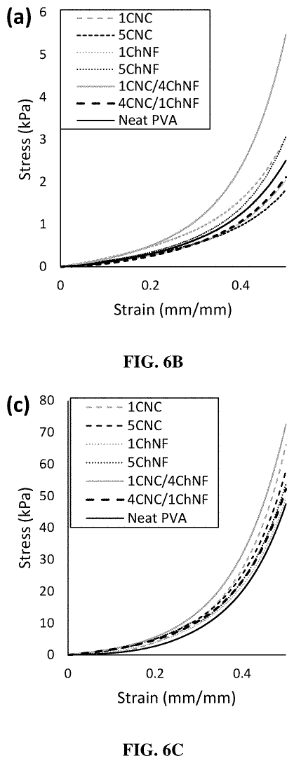

FIG. 8 depicts representative curves of aerogel samples that experienced three distinct compression phases identified by phase 1 box, phase 2 box, and phase 3 box.

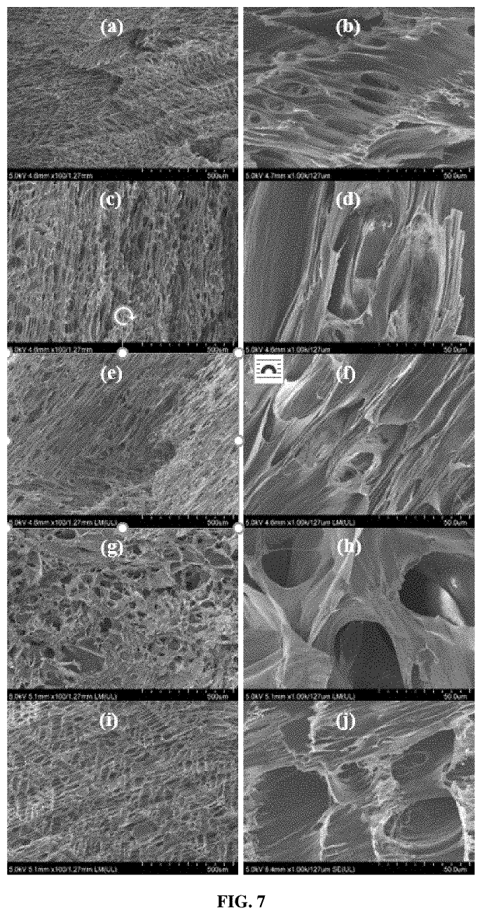

FIGS. 9A-9D depict mechanical properties of neat and composite aerogels including (FIG. 9A) modulus, (FIG. 9B) yield stress, (FIG. 9C) stress at 40% strain, and (FIG. 9D) stress at 80% strain.

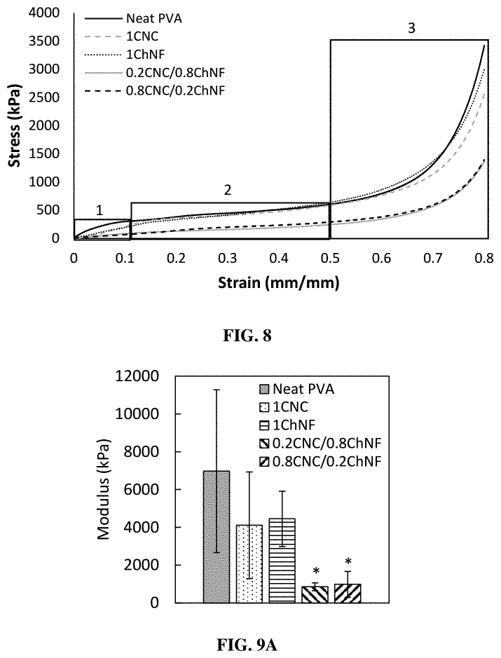

FIG. 10 depicts an IVD diagram showing a side view between two vertebrae in the spine and an alternate three-dimensional view showing the nucleus pulposus (NP) and annulus fibrosus (AF).

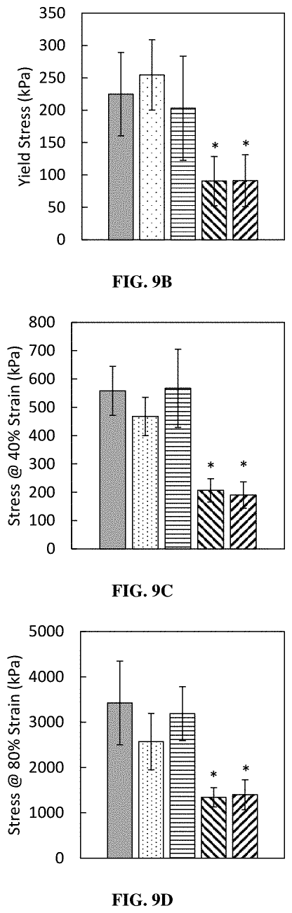

FIG. 11 depicts a CNC/ChNF/PVA hydrogel/aerogel hybrid.

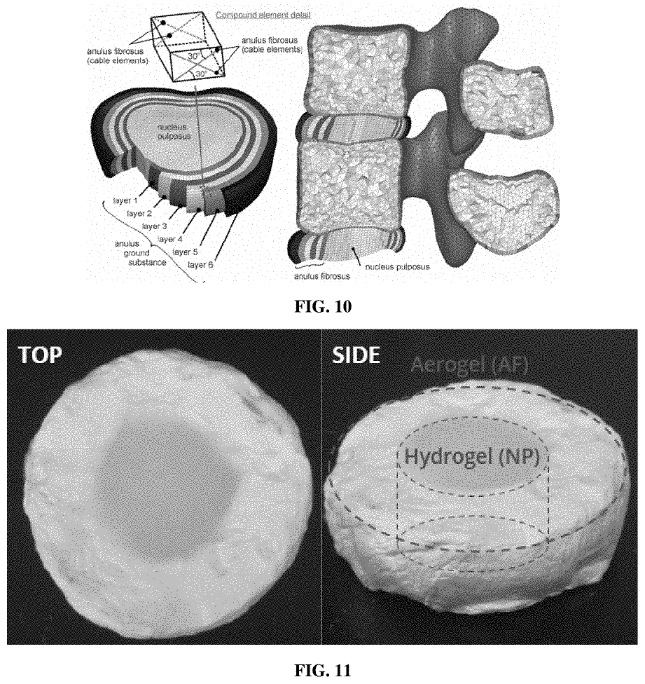

FIG. 12 depicts a stress-strain curve of an artificial intervertebral disc.

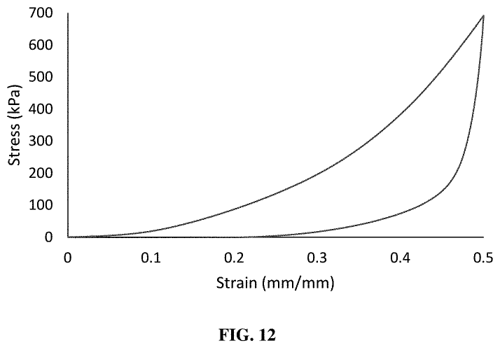

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Certain aspects of the invention relate to a multi-construct aerogel/hydrogel hybrid polymeric material, in some embodiments, includes poly(vinyl alcohol) (PVA). In some embodiments, the exemplary implant device includes an aerogel component (e.g., produced through a freeze-drying process) and a hydrogel component (e.g., in the center of the aerogel) (e.g., produced through freeze-thawing methodology).

In some embodiments, the base constituent materials include only PVA and water with no additional crosslinking compounds, or chemicals. The size and shape of the device and aerogel/hydrogel components can be modulated easily with custom molds. The aerogel and hydrogel components may be firmly connected. Mechanical response to compression combine the stiffness of the aerogel component with the elastic response of the hydrogel component. In some embodiments, nanomaterials may be added to further modulate the properties, such as biocompatibility.

In some embodiments, the exemplary device is configured for use as a biomimetic intervertebral disc replacement (or any other fibrocartilage replacement). The exemplary device may be configured for any modifiable compressive device.

Implantable device may include, or configured as, sections of intervertebral disc, as various joints around, or in, the knees, femurs, shoulders, joints, spine, head, jaw, hand, foot, elbows, ribs, etc.

In some embodiments, the construction of the device beneficially only require poly(vinyl alcohol) (PVA) and water. The device may include a PVA aerogel produced through freeze-drying of a frozen high molecular weight PVA solution. The aerogel may have a center carved out and replaced with a PVA hydrogel that is made up of PVA solution dispensed in the center and then subjected to physical crosslinking, e.g., through freezing and thawing. The two components are strongly adhered to one another to form a unitary body having a mechanical response to stress, pulls, shock absorption, predominantly from the hydrogel and general stiffness from the aerogel.

In another aspect, polymers that are structurally enhanced with cellulose and chitin based nanofillers are disclosed. While nanofillers can be included in extruded mixtures, their relatively low stability temperature makes solution processing a better route. PVA is highly water-soluble and may work well with dispersing fillers in solution and molding into various forms. The outer fibrous ring and vertebral endplates may be generated through casting of aqueous solution. Molds may be generated in specific shapes and dimensions and subsequently filled with a gel-like PVA solution for casting. The addition of chitin/cellulose nanofillers may improve the physical properties of the polymer and allow them to be finely tuned to the surrounding moduli based on ratios. The inner, softer section of the vertebrae may be generated with PVA hydrogels, which has been used previously in this application, but without the use of nanofillers for individualized customization. Lastly, a sheath developed by PVA foams maybe used to surround the complex. The compression strength of these foams may be alterable with the introduction of nanofillers up to certain loadings.

A study may be conducted to assess the effect of processing on the final structure. Crystallization may be assessed through conventional and Flash DSC, the latter being better suited for rapid crystallization, with optical microscopy being used to visually show crystal growth and directionality. Understanding this is important as direction of crystal growth can influence direction of cell growth. Many mechanical properties may be tested, e.g., via Instron, specifically compression strength, strain capacity, modulus, and fatigue life. For any cartilage tissue, the material's ability to withstand physiological compression forces over time with minimal degradation may be most important. These properties may be compared to those of in vivo requirements reported in literature and currently utilized spinal fusion and artificial disc devices.

In some embodiments, an integrated cellulose and chitin nanocrystals and fibers polymer composite may be used for artificial disc replacement that performs similarly to that of natural collagen discs. A total disc replacement of PVA solid outer fibrous ring, inner gel-like center, and foam sheath containing cellulose/chitin fibers and crystals will adhere strongly to the surrounding tissue while allowing for dynamic rotational and compressive movement. This three-part complex may serve as an artificial replacement that may be applicable to intervertebral discs, as well as other regions of degenerated fibrocartilage to improve comfort and overall quality of life for patients. Characterization of the material may be performed, e.g., through mechanical, chemical, and biological tests to assess the processing-structure-property relationship of nanocomposites in other applications. For example, the soft polymer foam has been previously suggested to be of lower production cost than that of conventional polyurethane.

In some embodiments, the construction of the device beneficially only require poly(vinyl alcohol) (PVA) and water. The device may include a PVA aerogel produced through freeze-drying of a frozen high molecular weight PVA solution. The aerogel may have a center carved out and replaced with a PVA hydrogel that is made up of PVA solution dispensed in the center and then subjected to physical crosslinking, e.g., through freezing and thawing. The two components are strongly adhered to one another to form a unitary body having a mechanical response to stress, pulls, shock absorption, predominantly from the hydrogel and general stiffness from the aerogel.

The hydrogels disclosed herein can have a density from about 0.50-1.5 mg/mm³, more preferably from 0.75-1.25 mg/mm³, and especially preferably from 0.9-1.1 mg/mm³. The aerogels disclosed herein can have a density that is no greater than 0.25 mg/mm³, no greater than 0.20 mg/mm³, no greater than 0.15 mg/mm³, no greater than 0.10 mg/mm³, no greater than 0.05 mg/mm³. In some embodiments, the aerogel can have a density that is from 0.05-0.25 mg/mm³, from 0.05-0.15 mg/mm³, or from 0.05-0.10 mg/mm³.

In certain embodiments, the PVA used for the hydrogel can have a molecular weight of at least 50,000 Da, at least 75,000 Da, at least 100,000 Da, at least 125,000 Da, at least 150,000 Da, at least 200,000 Da, or at least 250,000 Da. In some embodiments, the PVA used for the hydrogel can have a molecular weight from 50,000-500,000 Da, from 100,000-500,000 Da, from 125,000-500,000 Da, from 150,000-500,000 Da, from 175,000-500,000 Da, from 200,000-500,000 Da, from 50,000-500,000 Da, from 50,000-300,000 Da, from 50,000-250,000 Da, from 50,000-200,000 Da, from 75,000-250,000 Da, from 75,000-150,000 Da, from 75,000-125,000 Da, from 100,000-175,000 Da, or from 125,000-175,000 Da.

In certain embodiments, the PVA used for the aerogel can have a molecular weight of at least 50,000 Da, at least 75,000 Da, at least 100,000 Da, at least 125,000 Da, at least 150,000 Da, at least 200,000 Da, or at least 250,000 Da. In some embodiments, the PVA used for the hydrogel can have a molecular weight from 50,000-500,000 Da, from 100,000-500,000 Da, from 125,000-500,000 Da, from 150,000-500,000 Da, from 175,000-500,000 Da, from 200,000-500,000 Da, from 50,000-500,000 Da, from 50,000-300,000 Da, from 50,000-250,000 Da, from 50,000-200,000 Da, from 75,000-250,000 Da, from 75,000-150,000 Da, from 75,000-125,000 Da, from 100,000-175,000 Da, or from 125,000-175,000 Da.

In certain embodiments, the PVA used for the hydrogel can have a degree of hydrolysis of at least 95%, at least 97%, 98%, at least 99%, or at least 99.5%. In some embodiments, the PVA used for the hydrogel can have a degree of hydrolysis from 95-99%, from 97-99%, or from 98-99%.

In certain embodiments, the PVA used for the aerogel can have a degree of hydrolysis of at least 95%, at least 97%, 98%, at least 99%, or at least 99.5%. In some embodiments, the PVA used for the aerogel can have a degree of hydrolysis from 95-99%, from 97-99%, or from 98-99%.

In certain embodiments, the hydrogel and aerogels disclosed herein include PVA and a filler, the filler containing at least one of crystalline nanocellulose (“CNC” and chitin nanofibers (“ChNF”). In certain embodiments, the filler is solely crystalline nanocellulose, while in other embodiments, the filler is solely ChNF chitin nanofibers. When mixtures of the two components are used, the weight ratio of crystalline nanocellulose to chitin ChNF can be from 100:1-1:100, from 100:1 to 1:1, from 1:1 to 1:100, from 5:1 to 1:5, from 2.5:1 to 1:2.5, from 1:1 to 1:5, or from 5:1 to 1:1.

The hydrogels disclosed herein can include PVA and a filler, said filler present in an amount of at least 1 wt. %, least 2.5 wt. %, least 5 wt. %, least 7.5 wt. %, least 10 wt. %, least 15 wt. %, least 25 wt. %, least 30 wt. %, or at least 50 wt. %, relative to the total weight of the hydrogel. In some embodiments, the filler can be present in an amount from 1-50 wt. %, from 2.5-50 wt. %, from 5-50 wt. %, from 7.5-50 wt. %, from 10-50 wt. %, from 25-50 wt. %, from 1-25 wt. %, from 1-15 wt. %, from 1-10 wt. %, from 1-5 wt. %, from 5-15 wt. %, from 5-25 wt. %, from 15-25 wt. %, from 20-30 wt. %, or from 30-50 wt. %, relative to the total weight of the hydrogel.

The aerogels disclosed herein can include PVA and a filler, said filler present in an amount of at least 1 wt. %, least 2.5 wt. %, least 5 wt. %, least 7.5 wt. %, least 10 wt. %, least 15 wt. %, least 25 wt. %, least 30 wt. %, or at least 50 wt. %, relative to the total weight of the aerogel. In some embodiments, the filler can be present in an amount from 1-50 wt. %, from 2.5-50 wt. %, from 5-50 wt. %, from 7.5-50 wt. %, from 10-50 wt. %, from 25-50 wt. %, from 1-25 wt. %, from 1-15 wt. %, from 1-10 wt. %, from 1-5 wt. %, from 5-15 wt. %, from 5-25 wt. %, from 15-25 wt. %, from 20-30 wt. %, or from 30-50 wt. %, relative to the total weight of the aerogel.

The hydrogels can include a solvent, either water alone or in combination with one or more additional solvents. The water can be present in an amount of at least 50 wt. %, at least 60 wt. %, at least 70 wt. %, at least 80 wt. %, at least 90 wt. %, at least 95 wt. %, or at least 98 wt. %, relative to the total weight of the hydrogel.

The aerogels generally do not include a solvent. For example, the aerogel can include no more than 25 wt. %, no more than 20 wt. %, no more than 15 wt. %, no more than 10 wt. %, no more than 7.5 wt. %, no more than 5 wt. %, no more than 2.5 wt. %, no more than 1 wt. %, or essentially no solvent at all, relative to the total weight of the aerogel.

The crystalline nanocellulose may have an average first dimension (as measured by atomic force microscopy) of 10-1,000 nm, of 10-500 nm, of 10-100 nm, of 50-500 nm, of 50-250 nm, or of 100-250 nm. The crystalline nanocellulose may have an average second dimension (as measured by atomic force microscopy), of 0.5-25 nm, of 1-25 nm, of 1-20 nm, of 1-15 nm, of 1-10 nm, of 1-5 nm, of 5-10 nm, or of 5-15 nm.

The ChNFs may have an average first dimension (as measured by atomic force microscopy) of 100-2,000 nm, of 250-2,000 nm, of 500-2,000 nm, of 750-2,000 nm, of 1,000-2,000 nm, of 100-500 nm, 100-1,000 nm, of 100-1,500 nm, of 250-1,000 nm, or of 250-750 nm. The ChNF may have an average second dimension (as measured by atomic force microscopy), of 0.5-25 nm, of 1-25 nm, of 1-20 nm, of 1-15 nm, of 1-10 nm, of 1-5 nm, of 5-10 nm, or of 5-15 nm.

The compositions disclosed herein may be prepared by first dissolving PVA in water, optionally in combination with a water-soluble organic solvent in an amount not more than 25%, not more than 20%, not more than 15%, not more than 10%, or not more than 5% of the total solvent, by volume. Suitable water-soluble organic solvents include alcohols like methanol, ethanol, and propanol; acetone, acetonitrile, dioxane, and the like. The dissolution may be carried out using heat, for instance at a temperature above 30° C., above 50° C., about 60° C., or above 80° C.

The PVA solution is then brought to a temperature no greater than about 75° C., no greater than about 50° C., no greater than about 40° C., or no greater than about 30° C., and in some embodiments adding a small amount of acid. Suitable acids include lower alkyl acids, like acetic acid, butyric acid, maleic acid, succinic acid, and the like. The filler can be added to the PVA solution with mixing. For embodiment in which both crystalline nanocellulose and ChNFs are present in the filler, the crystalline nanocellulose may be added first, the ChNFs can be added first, or a pre-mixed combination of crystalline nanocellulose and ChNFs can be added.

The resulting mixture may be converted to a film using solution casting. The mixture may be converted to a hydrogel by subjecting the solution to at least one freeze/thaw sequence. The sequence may be repeated 1, 2, 3, 4, 5, 6, or more times in order to obtain a hydrogel with desired properties. Aerogels may be obtained by subjecting the resulting hydrogel to a lyophilization process. In certain embodiments, the aerogel is obtained by lyophilizing a hydrogel, wherein said hydrogel was subjected to no more than 1, 2, or 3 freeze/thaw cycles.

### EXAMPLES

The following examples are for the purpose of illustration of the invention only and are not intended to limit the scope of the present invention in any manner whatsoever.

### Example 1—Hybrid Films

Polyvinyl alcohol purchased from Sigma-Aldrich (St. Louis, Mo.) and used as received. LPVA=weight average MW 31,000-50,000 g/mol, 98-99% hydrolysed; HPVA=weight average MW 146,000-186,000, 99+% hydrolysed.

CNCs were derived from dissolving pulp that was hydrolysed with 64% sulfuric acid, with the crystalline regions then separated out and through a series of dilutions, filtrations, centrifugations, and settling. The resulting material was freeze-dried and stored in opaque bags. These freeze-dried CNCs were then redispersed in water at 5.5 wt. % solids content using a Talboys model 134-1 overhead mixer set at 2000 RPM for at least 90 minutes.

Crab shells sourced from bio-waste shells were first thoroughly washed several times in deionized (DI) water and then ground into a fine powder with a commercial grinder. The ground crab shell powder was then refluxed at a temperature of 110° C. with 5 wt. % NaOH for 6 hours. The resulting solids were passed through a filter and washed with DI water until the pH of the water was 7. These solids were then held in a 7 wt. % HCl bath for 6 hours at lab conditions, filtered again, and washed with DI water until the pH of the wash water was 7. These acid-treated chitin solids were then refluxed with 5 wt. % NaOH for 48 hours at a temperature of 110° C., then again filtered and washed with DI water until the wash water pH was 7. The final product of these steps was a fine, white powder of purified chitin, which was then dried in an oven at 60° C. for 24 hours to remove any residual water. In order to extract the ChNFs from the chitin powder, the powder was redispersed in DI water at 0.5 wt. % and passed through a high-pressure homogenizer. Prior to the first pass, the pH of the suspension was adjusted to 3.0 using glacial acetic acid to encourage dispersion of the positively charged chitin molecules. Glacial acetic acid was purchased from Sigma-Aldrich and used as received. The homogenizer used in this thesis was a Mini DeBEE Homogenizer (BEE International, South Easton, Mass.). The first 20 passes of the chitin suspension were carried out at a pressure of 1034 bar and with a 0.2 mm nozzle. The final 10 passes were carried out at a pressure of 1516 bar and with a smaller 0.13 nozzle for a total of 30 passes. A water-cooled heat exchanger was utilized throughout the homogenization process in order to cool the shear-heated nozzle and resulting ChNF suspension to below 35° C.

PVA powder of one molecular weight (HPVA or LPVA) was mixed with water at 300 RPM with a stir bar in a water bath at 100° C. until no visible PVA particles were present. The amount of PVA powder mixed was consistent at 4.75 g, but the amount of initial water it was dissolved into varied depending on the amount of nanofillers needed, which always resulted in a final loading of 5 wt. % in suspension. If containing CNCs and/or ChNFs, the solution was cooled to below 50° C. and 1 mL glacial acetic acid for every 100 mL of solution was added to reduce the solution's pH and encourage dispersion of the ChNFs. The desired amount of ChNFs suspended in water at 0.5 wt. % were then added to the PVA solution, and the components were mixed at 300 RPM with a stir bar for at least 30 minutes. Finally, the desired amount of CNCs suspended in water at 5.5 wt. % was added and mixed at 300 RPM with a stir bar for at least 30 minutes. The resulting nanocomposite suspension was cast into a polystyrene Petri dish and covered with aluminum foil to prevent contamination during drying. For neat PVA polymer samples containing no nanofillers, PVA was dissolved in water with a stir bar at 300 RPM in a 100° C. water bath. The resulting solution was cast in a polystyrene Petri dish and allowed to dry in the same fashion as the nanocomposite samples. Drying to a solid film took between 7-12 days depending on the water content and PVA type. In all cases studied here, the filler loading in the nanocomposites was kept constant at 5 wt. %. This filler loading consisted of either only CNCs, only ChNFs, or different mixtures of CNCs and ChNFs at weight ratios of 1:4, 1:1, and 4:1. The naming convention for samples in this study follows the template: [wt. %]CNC/[wt. %]ChNF/[L or H]PVA. For example, a sample containing 1 wt. % CNC and 4 wt. % ChNF in high molecular weight (MW 146,000-186,000) PVA is denoted as 1CNC/4ChNF/HPVA.

For the titration testing, a 5.5 wt. % aqueous suspension of CNCs was diluted with DI water to approximately 1 wt. %. The resulting suspension was ion exchanged with Merck Ion Exchanger I, a strongly acidic cation exchange resin, to remove any cations. The resin was then washed with DI water to wash out entrapped CNCs and the resulting suspension was titrated against a 1.5 N NaOH solution by potentiometric titration with a Mettler Toledo Seven Excellence S400 pH meter. A 0.5 wt. % aqueous ChNF suspension was ion exchanged with Alfa Aesar Amberlite IRN-78, a strongly basic anion exchange resin, to remove acetate anions. This resin was then washed with DI water to wash out entrapped ChNFs. A volume of 20 ml of 0.25 N HCl was added to the resulting suspension which was then titrated against a 0.5 N NaOH solution by potentiometric titration. Both titrations were repeated three times, and the results were reported as an average±standard deviation.

For zeta-potential testing, a series of aqueous CNC/ChNF suspensions were prepared by mixing the 0.5 wt. % ChNF and 5.5 wt. % CNC aqueous suspensions to obtain different ratios of ChNF to CNC by weight. The suspensions were diluted with acidified water that consisted of 1 mL acetic acid in 100 mL water to mimic the nanocomposite preparation conditions. Each suspension's zeta-potential was then measured using a Malvern Nano-ZS90 Zetasizer with an equilibration time of three minutes. Each reading was repeated three times, and the count averaged zeta-potential of distribution was used with the maximum-observed standard deviation of the three readings being used as the uncertainty.

Samples were cut from the films with an ASTM D-1708 die cutter and dried for one hour in an oven at 110° C. to remove water. Samples were held on the ends with the center bridge suspended in air to optimize water removal uniformly from the testing region of the sample. Samples were then kept at laboratory conditions for 40-48 hours, and humidity levels in the laboratory were monitored. Humidity levels were observed to stay between 35% and 52% depending on the day of measurement.

Samples were tested following the ASTM D-1708 standard for polymer microtensile testing. An Instron 5566 Materials Testing Frame and a 1000 N load cell were used for testing tensile properties. After drying and conditioning, each sample was placed in the grips at a gage length of 22 mm. Samples' thicknesses were 0.3 mm±0.09 mm. The tests were conducted at a displacement rate of 0.1 mm/minute until fracture. While the microtensile testing standard is not designed to obtain quantitative Young's modulus values, the testing data were used to obtain relative modulus data. The relative modulus of the samples was calculated by taking the slope of the stress-strain curve from 5 MPa to 30 MPa for each sample. Tensile strength was indicated as the maximum stress experienced by the sample during testing. Strain at break values were calculated from the initiation point of 40% drop off in recorded force within the software. All data reported in this study is represented as an average±standard deviation. For statistical analysis between sets, a two-tailed Student's T-Test assuming unequal variances and an alpha value of 0.05 was performed. Statistical significance was determined by having a sample set average with a p value of less than 0.05. Sample set averages that were statistically significantly greater than the neat PVA film were indicated with an * in figures, while a \hat{ } indicates a sample set average that is statistically significantly greater than all other values.

Neat PVA and nanocomposite films were imaged with POM to qualitatively assess the levels of aggregation between nanofillers in each sample. Neat PVA films possessed a consistent coloring throughout. In comparison, the 5CNC samples showed white features that could be birefringence from CNCs aggregates. In contrast, the 5ChNF samples showed fewer white areas and resembled the neat polymer films more closely, possibly indicating that there was less aggregation of ChNFs. The tricomponent composites showed white features as well, and the features generally were smaller and more homogeneously distributed over the area in the images. Overall, these images indicated that some degree of nanofiller agglomeration was present in all of the nanocomposites containing CNCs. Despite the appearance of nanofiller aggregation in POM images, the neat PVA and nanocomposite films appeared transparent to the naked eye

Titration and zeta-potential testing were performed on the hybrid materials. The titration tests provided surface charge values for CNC and ChNF in aqueous suspension, while the zeta-potential tests investigated how these surface charges changed when CNC and ChNF suspensions were combined. The titration experiments yielded surface charge values of 1.4±0.1 meq/g and 0.49±0.09 meq/g for the ChNFs and CNCs, respectively.

The resulting equivalents for ChNFs and CNCs from these tests represented the maximum number of cationic groups, primarily free amine groups for ChNFs, and, anionic groups, primarily sulfate groups for CNCs that could participate in any neutralization reactions. When CNCs and ChNFs were mixed together in suspension, the oppositely charged surface groups could interact, leading to the formation of a ChNF-CNC complex or aggregate. However, this assembly process would be dependent on the total number of free groups present, which in turn would depend on the pH of the surrounding medium and presence of ions, as governed by the screening effect. The actual aggregate formation could be inferred from zeta potential measurements of various ChNF/CNC mixtures.

In CNC/ChNF mixtures, the suspensions were stabilized by strong electrostatic repulsions between oppositely charged particles. In suspensions containing CNCs and ChNFs, the zeta potential values were intermediate between those obtained for the suspensions containing only one type of nanofiller.

ATR-FTIR analysis was performed on both HPVA and LPVA for each of the five nanofiller loaded samples and neat polymer with the aim of understanding the changes in chemical structure as a result of the introduction of CNCs and ChNFs into the system. A large peak in the 3500-3000 cm⁻¹ range was attributed to stretching of hydrogen-bonded hydroxyl groups. Hydroxyl groups are present on PVA, CNC and ChNF, as well as any absorbed water that is present. The highest intensity peak in this range belonged to 2.5CNC/2.5ChNF/HPVA, followed by 1CNC/4ChNF/HPVA, with progressively smaller intensity peaks from 5ChNF/HPVA, 5CNC/HPVA, neat HPVA, and 4CNC/1ChNF/HPVA. Two peaks at 1720 and 1660 cm⁻¹ could be attributed to C═O and C—O stretching of acetyl groups, which appeared to grow in intensity with ChNF composition, as expected. The medium sized peak at 1410 cm⁻¹ was assigned to CH₂ and CH₃ bending deformation. The peaks around 1380, 1327, and 1235 cm⁻¹ were attributed to the bending of C—H, CH₂, and —OH, while the peak around 1086 cm⁻¹ was considered to be C-O stretching. There is an additional peak at 1065 cm⁻¹ that was only present in samples containing at least 2.5 wt. % CNCs, indicating that it may be the result of alkoxy C—O—C group stretching or primary aliphatic alcohol stretching in CNCs. However, despite the presence of alkoxy groups in ChNFs, this peak does not appear in the samples containing higher amounts of ChNFs. Skeletal signals appeared around the 915 and 845 cm⁻¹ bands.

In regard to the differences in intensities of the hydrogen bonding peak between 3500-3000 cm⁻¹, the highest intensity peak belonged to 2.5CNC/2.5ChNF/LPVA, followed by 5CNC/LPVA, 5ChNF/LPVA, 1CNC/4ChNF/LPVA, 4CNC/1ChNF/LPVA, and neat LPVA. Additionally, while only 4CNC/1ChNF/HPVA and 5ChNF/HPVA experienced a small shift at this peak to higher wavenumbers compared to neat HPVA, the entirety of the LPVA sample set experienced a 10-20 cm⁻¹ shift to higher wavenumbers in comparison to neat LPVA. While the peaks were broad, a shift to lower wavenumbers is often attributed to the presence of hydrogen bonds, which suggested the addition of CNCs/ChNFs to the LPVA caused a decrease in the amount of hydrogen bonding within the system. A main difference between the HPVA and LPVA sets was in regard to the 1720 cm⁻¹ peak, which was much more pronounced in the LPVA sample sets.

The table below outlines the two onset degradation events (OD-1, OD-2) measured using TGA, and residual weight percentage left in the pan at the conclusion of the test.

| | | | Residual |
|---|---|---|---|
| Sample Name | OD-1 (° C.) | OD-2 (° C.) | Weight (%) |
| Neat HPVA | 241 | 406 | 7.1 |
| 5CNC/HPVA | 253 | 400 | 10.3 |
| 4CNC/1ChNF/HPVA | 257 | 400 | 8.5 |
| 2.5CNC/2.5ChNF/HPVA | 262 | 429 | 8.9 |
| 1CNC/4ChNF/HPVA | 256 | 423 | 9.4 |
| 5ChNF/HPVA | 242 | 421 | 11.7 |
| Neat LPVA | 252 | 429 | 9.1 |
| 5CNC/LPVA | 256 | 412 | 8.2 |
| 4CNC/1ChNF/LPVA | 263 | 411 | 8.5 |
| 2.5CNC/2.5ChNF/LPVA | 248 | 414 | 9.5 |
| 1CNC/4ChNF/LPVA | 246 | 419 | 10.5 |
| 5ChNF/LPVA | 254 | 423 | 10.5 |

The table below displays the DSC-determined melting temperature, enthalpy of fusion, and % crystallinity values taken from the reversible heat flow signal during melting.

| | | Enthalpy of | |
|---|---|---|---|
| Sample Name | MP (° C.) | Fusion (J/g) | Crystallinity (%) |
| NeatHPVA | 211 ± 3 | 61 ± 0 | 38 ± 0 |
| 5CNC/HPVA | 200 ± 0 | 68 ± 0 | 44 ± 0 |
| 4CNC/1ChNF/HPVA | 199 ± 8 | 39 ± 9 | 25 ± 5 |
| 2.5CNC/2.5ChNF/HPVA | 198 ± 0 | 49 ± 1 | 32 ± 1 |
| 1CNC/4ChNF/HPVA | 200 ± 3 | 45 ± 7 | 29 ± 5 |
| 5ChNF/HPVA | 200 ± 0 | 40 ± 10 | 27 ± 6 |
| Neat LPVA | 200 ± 8 | 41 ± 3 | 25 ± 2 |
| 5CNC/LPVA | 200 ± 2 | 52 ± 8 | 34 ± 5 |
| 4CNC/1ChNF/LPVA | 187 ± 1 | 48 ± 4 | 31 ± 3 |
| 2.5CNC/2.5ChNF/LPVA | 188 ± 2 | 51 ± 2 | 33 ± 1 |
| 1CNC/4ChNF/LPVA | 190 ± 3 | 38 ± 4 | 25 ± 2 |
| 5ChNF/LPVA | 184 ± 1 | 49 ± 6 | 32 ± 3 |

Mechanical testing data consisting of modulus, tensile strength, and strain at break were collected from the neat PVA and nanocomposite samples that contained various ratios of CNCs and ChNFs. These data are shown in the table below.

| Sample (No. of | | Tensile | Strain at |
|---|---|---|---|
| Specimens) | Modulus (MPa) | Strength (MPa) | Break (%) |
| Neat HPVA (7) | 5210 ± 1040 | 112 ± 15 | 17.9 ± 9.2 |
| 5CNC/HPVA (7) | 6540 ± 458 | 128 ± 11 | 14.3 ± 10.5 |
| 4CNC/1ChNF/HPVA (5) | 6420 ± 710 | 116 ± 13 | 18.6 ± 25.4 |
| 2.5CNC/2.5ChNF/ | 6570 ± 427 | 121 ± 31 | 3.3 ± 1.6 |
| HPVA (6) | | | |
| 1CNC/4ChNF/HPVA (8) | 7430 ± 532 | 138 ± 7 | 7.0 ± 3.3 |
| 5ChNF/HPVA (9) | 6320 ± 763 | 130 ± 16 | 15.8 ± 9.6 |
| Neat LPVA (4) | 5200 ± 428 | 115 ± 11 | 3.3 ± 1.3 |
| 5CNC/LPVA (6) | 6550 ± 487 | 107 ± 12 | 2.1 ± 0.4 |
| 4CNC/1ChNF/LPVA (4) | 5670 ± 235 | 80 ± 11 | 1.6 ± 0.3 |
| 2.5CNC/2.5ChNF/ | 7470 ± 258 | 120 ± 7 | 1.9 ± 0.1 |
| LPVA (6) | | | |
| 1CNC/4ChNF/LPVA (6) | 6600 ± 557 | 108 ± 11 | 2.0 ± 0.3 |
| 5ChNF/LPVA (6) | 6510 ± 383 | 117 ± 4 | 2.4 ± 0.1 |

### Example 2: Hydrogel

PVA (146,000-186,000 g/mol, 99+% hydrolyzed) was dissolved in water in a water bath at 100° C. while stirring at 300 RPM with a stir bar until no visible PVA particles were present, typically 6 to 8 hours. The PVA solution was covered with aluminum foil during the duration of the mixing process to minimize evaporation and contamination. The initial content of water that the PVA was dissolved into varied depending on the weight loadings of CNCs and/or ChNFs to be added. The PVA solution was then cooled to below 50° C., and 1 mL glacial acetic acid for every 99 mL of polymer solution was added to encourage dispersion of the ChNFs. Then, the desired amount of ChNF suspension was added to the PVA solution, and the components were mixed at 300 RPM with a stir bar for at least 30 minutes. Finally, the desired amount of CNC suspension was added, and the nanocomposite suspension was mixed at 300 RPM with a stir bar for at least 30 additional minutes. Suspension volume was approximately 120 mL in all cases. After suspensions were prepared, they were sonicated in a Misonix Sonicator 3000 for five minutes at 80 W in order to improve dispersion of the nanofiller particles. For neat PVA solutions prepared for comparison, only the first mixing step was used.

Sample names in this document follow the following structure: [wt. %]CNC/[wt. %]ChNF. The weight percentages correspond to the loading in the solid phase of the suspension. This naming convention was also used previously for nanocomposite film samples produced by the authors. To demonstrate this naming convention, a sample containing 1 wt. % CNC and 4 wt. % ChNF is denoted as 1CNC/4ChNF, while a sample containing only 1 wt. % CNC is denoted as 1CNC. Nanofiller loadings of 1 wt. % and 5 wt. % were prepared to test differences between loadings of CNCs 1CNC and 5CNC) and. ChNFs (i.e. 1ChNF and 5ChNF) in addition to two tricomponent samples of 1CNC/4ChNF and 4CNC/1ChNF chosen based on their performance from a previous study. Additionally, using the sizes and densities of the nanomaterials, it was approximated that a 1:1 number ratio between CNCs and ChNFs would require 27% more ChNFs than CNCs. For a composite containing 5% total nanofillers, a 1.4CNC/3.6ChNF/PVA composite would have approximately one CNC for every ChNF. Therefore, the 1CNC/4ChNF sample would containing more ChNFs than CNCs, and vice versa for the 4CNC/1ChNF sample.

The resulting polymer solution or nanocomposite suspension was poured into an aluminum mold containing 20 cylindrical wells with a height of 12 mm and a diameter of 24 mm. The aqueous solution/suspension in the aluminum mold was then placed in a freezer at −10° C. to freeze the samples and allow for the formation of physical crosslinks. Throughout this document, any mention of crosslinks are physical in nature. Samples remained in the freezer for approximately 16 hours before being removed and allowed to thaw for approximately eight hours at ambient laboratory conditions. This 24-hour process constitutes one complete FT cycle and was repeated up to seven times.

Cylindrical hydrogel samples were divided into four parts and submerged in 50 mL water and weighed over the course of a six-hour period with timepoints at 0, 10, 20, 30, 45, 60, 75, 90, 105, 120, 180 240, 300, and 360 minutes. At the specified time points, the samples were vigorously shaken for three seconds when removed from the water bath to remove surface water then weighed. After each measurement, samples were placed back into the water for subsequent measurements. These experiments aimed to develop understanding of how these materials behave in specific environments and elucidate the water retention properties of the hydrogels after various FT cycles. Statistical analysis of comparisons between sets for this and all other analyses in this study were performed with two-tailed Student's T-Tests assuming unequal variances and an alpha value of 0.05.

Mechanical properties were assessed through compression testing of the samples at a constant displacement rate. These results were used to understand the effects of the nanofillers, their mixtures, and the number of FT cycles on the hydrogels' properties. While not specific for hydrogel samples, ASTM D1621-16: Standard Test Method for Compressive Properties of Rigid Cellular Plastics was used to specify a sample geometry. The standard recommends a maximum height to diameter ratio of 1:1, so a height of 10 mm and diameter of 24 mm were chosen for these tests. Freezing would cause greater expansion in some samples compared to others, so sample heights were slightly varied (i.e. ±1 mm). Additionally, the cross sections of the 7FT samples were distorted to an irregular oval shape, so diameters were reported as an average of four measurements around the sample. Prior to testing, a preload of 0.01 N was applied to the 1FT sample, and a preload of 0.1 N was applied for the remaining samples. The testing speed used was 1.2 millimeters per minute. Compression tests were run with n=4 or 5 to 50% compression, and the average stress achieved at 50% strain was reported with standard deviations. Average modulus was calculated as the slope of the stress-strain curve over a strain range of 0 to 3% and reported with standard deviation.

The appearance of the hydrogels was altered by repeated FT cycling, but remained similar for neat and composite samples. After 1FT cycle, the hydrogels were gelatinous and somewhat translucent. After 3FT cycles, the hydrogel samples became more opaque and whiter in color and were observed to be more rigid during handling. The appearance after 5FT cycles was similar to that observed after 3FT cycles. After 7FT cycles, the hydrogel samples began to shrink slightly in the aluminum mold, likely due to increased and/or more robust network junctions.

The water absorption values were affected by hydrogel composition and number of FT cycles. Overall, the amount of mass change decreased or became negative as the number of FT cycles increased, consistent with a more rigid structure. Many of the hydrogels exposed to higher numbers of FT cycles experienced some increase in mass at short submersion times, even if the mass change value recorded after six hours was negative. Comparatively, the 5ChNF hydrogel absorbed more water after 1FT and retained more water after 5FT and 7FT compared to the neat PVA hydrogel. However, the 5ChNF hydrogel did not experience as large of an initial mass change as the neat PVA.

After 1FT cycle, all hydrogels increased in mass. It was considered likely that the level of cros slinking would be relatively low; therefore, expansion of the molecular network upon exposure to water was expected. The sample composition for the 1FT hydrogels affected the magnitude of water absorption, with the 5CNC hydrogel having the smallest increase in mass and the 5ChNF hydrogel having the largest increase in mass. The neat PVA had a mass increase value between these two extremes. The increased level of crosslinking in the 3FT hydrogels was expected to generate a more rigid structure, impeding their ability to absorb water and resulting in a lower maximum water absorption compared to 1FT hydrogels. Four 3FT hydrogels had minimal or slightly negative mass changes: 1CNC, 5CNC, 1ChNF, and 4CNC/1ChNF. For 5FT and 7FT hydrogels, mass changes after six hours for all hydrogels except 5ChNF were negative.

The FT process for producing PVA hydrogels generated a network structure made up of polymer chains interconnected through hydrogen bonding and polymer crystallites along the perimeter of pores. The distance between network junction points, or mesh size, can be influenced by adjusting the processing parameters or adding fillers to the PVA matrix. While it can be challenging to confirm the pore size/structure of a hydrogel, particularly when fillers are introduced, aerogels produced through freeze-drying of PVA hydrogels have previously demonstrated that nanofillers can lead to the pore shrinkage. Due to this, it is possible that within the hydrogel structure there is a similar shrinkage of pores with varied levels of junction reinforcement as a result of the interactions between nanofillers and the matrix.

The different response of the 5ChNF hydrogel in comparison to the other samples suggested that the component interactions were different in this hydrogel. Additionally, the similarity in behavior between neat PVA hydrogels and hydrogels containing CNCs could indicate that intermolecular interactions were similar and stronger than those seen between ChNFs and PVA. The expected hydrogen bonding interactions between CNCs and the PVA molecules could result in a more rigid pore structure that would constrict and expel water in response to changes in external pressure experience when submersed. Conversely, the increased absorption/retention of water in ChNF-loaded hydrogels suggested that the degree of hydrogen bonding could be less in comparison to CNC-loaded hydrogels, which would likely result in a less rigid structure.

Elastic modulus was found to vary with the number of FT cycles and with hydrogel composition. For each hydrogel composition, the modulus increased as the number of FT cycles increased from 1FT to 3FT. Going from 3FT to 7FT, the modulus of 5ChNF, 1CNC/4ChNF, and 4CNC/1ChNF increased. Other compositions did not display statistically significant increases when comparing 7FT samples to 3FT samples. Comparing the modulus values of different compositions for a given number of FT cycles, only the 1CNC/4ChNF was found to have a statistically larger modulus than the neat PVA after 1 FT cycle. Additionally, the modulus of this hydrogel was larger than all of the other hydrogels tested, demonstrating that tricomponent composite hydrogel can have higher stiffness than composite hydrogels containing only one of the filler types at the same overall filler loading. This result for 1CNC/4ChNF was qualitatively consistent with the reinforcement observed for tricomponent film samples. However for the films, other composites also had higher moduli than the neat PVA films, suggesting some differences in the mechanical reinforcement between the film construct and hydrogel construct. For 3FT cycles, all of the hydrogels had statistically similar modulus values in comparison to the neat PVA hydrogel. This result showed that the increased phase separation and network development had a larger impact than composition at 3FT cycles. Considering the modulus values obtained for 7FT hydrogels, the 3FT hydrogels represented a transitional state in the structure development because some composite hydrogels prepared with 7FT cycles did have higher moduli values than the neat PVA. Specifically, 4CNC/1ChNF, 1CNC/4ChNF, and 5ChNF hydrogels had higher moduli than the neat PVA hydrogel. This result differed from the elastic modulus results obtained for films. In that work, all of the composites at a 5 wt. % filler loading had a higher modulus than the neat PVA.

Like elastic modulus, maximum compressive stress was found to vary with number of FT cycles and hydrogel composition. For a given hydrogel composition, the maximum compressive stress increased as the number of FT cycles increased. This trend was consistent with an increase in crosslinking within the samples, as shown previously in the swelling studies. The hydrogels experienced larger percentage increases between 1FT and 3FT, and smaller percentage increases between 3FT and 7FT. With regard to hydrogel composition, all hydrogels except 1CNC had a statistically different value of the maximum compressive stress as compared to the neat PVA at 1FT, though only 1CNC/4ChNF and 5ChNF had larger values than neat PVA. At 3FT, three samples had larger values of maximum compressive stress than the neat PVA. These samples were 1CNC, 1CNC/4ChNF, and 5ChNF. This result was different than that seen for elastic modulus at 3FT. At 3FT, the composite hydrogels had similar modulus values to neat PVA. This effect was not seen as widely at 7FT, where only the 1CNC/4ChNF hydrogel had a larger maximum compressive stress than the neat PVA.

| | 1FT | 3FT | 7FT |
|---|---|---|---|
| Sample | Modulus/Stress (kPa) | Modulus/Stress (kPa) | Modulus/Stress (kPa) |
| Neat PVA | 0.78 ± 0.12/2.49 ± 0.13 | 6.15 ± 1.8/23.2 ± 3.7 | 5.90 ± 5.0/53.0 ± 10.9 |
| 1CNC | 0.95 ± 0.52/2.79 ± 0.44 | 5.36 ± 0.77/28.3 ± 1.1* | 9.37 ± 6.9/69.5 ± 13.8 |
| 5CNC | 0.89 ± 0.48/1.69 ± 0.43* | 8.55 ± 2.0/21.3 ± 8.3 | 10.61 ± 4.0/60.1 ± 11.2 |
| 1ChNF | 0.91 ± 0.28/2.01 ± 0.30* | 6.57 ± 0.69/22.7 ± 1.1 | 9.91 ± 4.4/53.6 ± 7.0 |
| 5ChNF | 0.84 ± 0.25/3.31 ± 0.57* | 7.11 ± 1.5/30.0 ± 1.0* | 14.54 ± 5.2*/58.3 ± 6.6 |
| 1CNC/4ChNF | 1.81 ± 015*/5.68 ± 0.54* | 6.92 ± 1.7/29.6 ± 1.7* | 15.38 ± 2.1*/71.5 ± 2.2* |
| 4CNC/1ChNF | 0.82 ± 0.39/2.07 ± 0.17* | 6.30 ± 0.78/20.5 ± 1.0 | 12.46 ± 1.2*/52.1 ± 1.0 |

### Example 3: Aerogel

PVA (146,000-186,000 g/mol, 99+% hydrolysis) was dissolved in water while being mixed with a stir bar at 300-750 RPM in a water bath at 100° C. for eight hours or until no visible PVA particles were present. For nanocomposite samples, the solution was cooled to below 50° C. and acetic acid was added to achieve a 1 vol.% concentration to protonate the solution and encourage the dispersion of ChNFs. A 0.5 wt. % ChNF suspension was next deposited into the solution and mixed at 750 RPM with a stir bar for at least 30 minutes. Lastly, for composites containing CNCs, a 0.5 wt. % solution of CNCs was added to the PVA solution or ChNF/PVA suspension and mixed at 750 RPM with a stir bar for at least 30 minutes. The final total solids loading of all suspensions was 5 wt. %. Nanocomposite solutions were sonicated in a Misonix Sonicator 3000 for five minutes at 80W to break up larger aggregates and encourage nanofiller dispersion. 20 mL of the prepared solutions were syringed into 20 mL cylindrical glass scintillation vials of approximately 24 mm in diameter and 45 mm in height. The glass vials were placed in a −10° C. freezer and frozen overnight for approximately 18 hours before being removed. The glass vials were then wrapped and broken, with the intact hydrogels removed in order to expose all parts of the sample and maximize the amount of surface area available for sublimation. Lastly, the frozen samples were placed in individual glass beakers in a freeze-dryer at approximately −50° C. and 0.1 mBar pressure for three days to sublimate water out of the neat and composite samples. Resulting aerogels were cut with a razor blade to generate cylindrical samples of approximately 10 mm in height for compression testing, with 5 mm portions from the center cut out and set aside for scanning electron microscopy (SEM) analysis. The naming convention for samples in this study use the following structure: [wt. %]CNC/[wt. %]ChNF. Nanocomposite samples were kept at a consistent 1 wt. % loading with five total sample groups: 1CNC, 1ChNF, 0.2CNC/0.8ChNF, 0.8CNC/0.2ChNF, and Neat PVA. T

Neat and nanocomposite aerogel samples approximately 10 mm in height and 18 mm in diameter were used for compression testing to assess the impact of the CNCs and ChNFs on the mechanical behavior. The samples were cut from the as prepared aerogels from the top to the bottom with a single cylinder yielding up to three samples. The individual samples were then weighed, and the diameter was taken from the average of four measurements. The height was determined from the Instron prior to running the compression testing by lowering the compression head until a 0.1 N force was registered. These measurements were used to calculate the density of the samples with the following equation:

$$ρ = \frac{m}{( d π h )}$$

In this equation, ρ is the density, m is the mass, d is the diameter, and h is the height of the sample. The samples were assumed to be cylindrical for volume measurements, and the density of each sample set was compared to one another.

Compression testing was performed by applying a 10 N preload to the samples and then compressing at 10% strain per minute up to 80% strain. The modulus was measured as the slope of the stress-strain curve over the initial 1% strain, and the energy associated with deformation was measured as the area under the stress-strain curve. The solids modulus and solids stress were also calculated in order to adjust the measured values to the differences in densities of the neat and composite materials. The values were calculated from a method described by Gibson and Ashby (1982) and were based on the relative density of the materials, or the ratio of aerogel density to solid density. Below is the formula for calculating these two values:

$$\frac{{E}^{*}}{{E}_{s}} = {( \frac{{ρ}^{*}}{{ρ}_{s}} )}^{2}$$

$$\frac{{σ}_{pl}^{*}}{{σ}_{ys}} = 0.3 {( \frac{{ρ}^{*}}{{ρ}_{s}} )}^{\frac{3}{2}}$$

In these equations, E* is the aerogel modulus, Eₛ is the moduli for cell wall material, ρ* is the aerogel density, ρₛ is the solids density, σ*ₚₗ is the aerogel stress at yield, and σ_{ys} is the solids yield stress. The density of the solids was calculated as a ratio of the individual weight loadings and densities of the components in each sample set. To compare values of each set, the median was chosen from the data sets. The yield stress was determined by calculating the stress value at the intersecting strain value between the elastic modulus and the linear trend line calculated from the 20-23% plateau range of the curves. Yield stress values calculated with this methodology that either occurred at strain values larger than 20% or less than 0% strain were not considered and those curves' values were manually chosen based on the point of greatest slope change between 0-20%.

Density was first measured in order to assess the level of macro-scale structural shrinkage that the nanofillers may have caused during the freeze-drying process. Neat PVA had the highest average density of the sample sets at 138 kg/m³, which was evident by the smaller diameters of the neat PVA aerogels relative to the nanocomposite samples. Conversely, 1CNC had the lowest density of the tested samples at 99 kg/m³, while samples containing any amount of ChNFs had density values that were between neat PVA and 1CNC aerogels at about 110 kg/m³.

To analyze the effects of the nanofillers on the mechanical properties of the aerogels, samples were compressed between two plates to a total strain of 80% and various values were extracted from the resulting stress-strain curves.

The first stage consisted of linear elastic deformation before plateauing around 10% strain. In this stage, neat PVA possessed the highest slope between 0% and 10%, followed by 1CNC, 1ChNF, 0.2CNC/0.8ChNF, and 0.8CNC/0.2ChNF. The second stage involved plastic yielding as a result of pore cracking in which the pores of the sample would continually collapse under the force of the compression and limit any increase in recorded stress. This stress plateau would continue until about 40% strain at which point the pore structure would be completely collapsed. The neat PVA, 1CNC, and 1ChNF curves exhibit a clear yield point and transition between phases 1 and 2, whereas the tricomponent curves appear to experience a lesser difference in slope change as they hit their plateau. This qualitative difference can be inferred as the tricomponent samples quickly experiencing pore cracking in response to compression with little elastic response. The third stage consisted of an exponentially increasing stress-strain curve as the collapsed pores would densify the polymer structure. All samples exhibited similar shapes.

While the bulk property values provided information about the overall structure of the PVA aerogels, the density could be used to adjust mechanical properties for a more complete understanding of how the nanofillers may be reinforcing or weakening the PVA matrix structure on the nanoscale. In order to adjust the density, the solids density of the constituent materials first needed to be calculated. The solids densities of each material were calculated based on the volume and densities of the constituent materials where the density of PVA was 1260 kg/m³, the density of CNCs was 1600 kg/m³, and the density of ChNFs was 1425 kg/m³. The resulting solids densities were 1260 kg/m³ for neat PVA, 1263 kg/m³ for 1CNC, 1261 kg/m³ for 1ChNF, and 1262 kg/m³ for both 0.2CNC/0.8ChNF and 0.8CNC/0.2ChNF.

Density, pore size, and mechanical properties of neat PVA and composite samples. Values are reported as averages±standard deviations.

| | | | | | Energy of |
|---|---|---|---|---|---|
| | Density | Pore Area | | | Deformation |
| | (kg/m³) | (nm²) | E (MPa) | σ_{y} (kPa) | (kPa) |
| Neat PVA | 138 ± 15 | 255 ± 66 | 6.98 ± 4.3 | 225 ± 64 | 620 ± 120 |
| 1CNC | 99 ± 9.9* | 972 ± 320* | 4.11 ± 2.8 | 255 ± 54 | 509 ± 91 |
| 1ChNF | 113 ± 8.0* | 575 ± 200* | 4.45 ± 1.5 | 203 ± 81 | 614 ± 130 |
| 0.2CNC/0.8ChNF | 109 ± 6.3* | 1240 ± 420* | 0.860 ± 0.21* | 90.6 ± 38* | 234 ± 44* |
| 0.8CNC/0.2ChNF | 113 ± 13* | 1190 ± 320* | 0.985 ± 0.68* | 91.3 ± 40* | 224 ± 51* |

| | E (MPa) | σ_{y} (kPa) | Eₛ (MPa) | σ_{ys} (MPa) |
|---|---|---|---|---|
| Neat PVA | 5.91 | 214 | 460 | 19.1 |
| 1CNC | 3.27 | 255 | 629 | 39.1 |
| 1ChNF | 4.27 | 204 | 540 | 26.1 |
| 0.2CNC/0.8ChNF | 0.818 | 82.5 | 114 | 10.5 |
| 0.8CNC/0.2ChNF | 0.881 | 85.5 | 106 | 11.1 |

### Example 4: Medical Implant

Intervertebral discs (“IVDs”) is a relatively simple structure composed of two major components: (1) an outer fibrous set of lamellar rings called the annulus fibrosus (AF) and (2) a gel-like material at the center called the nucleus pulposus (NP). The stiffer AF takes the bulk of the load in most situations, while the NP acts a kind of shock absorber for instant-impact forces.

An artificial IVD was constructed with a composite aerogel serving as the AF and a composite hydrogel served as the NP. A 1CNC/PVA solution with 95% water was prepared as outlined above with the suspension being poured into a 75 mm diameter and 26 mm high Teflon mold and frozen for 16 hours. The frozen 1CNC/PVA hydrogel was then placed into a freeze-dryer and allowed to sublimate for three days to remove water. After the freeze-drying process was completed, a one-inch diameter die was used to cut out a hole from the center of the aerogel. This hole was filled with a prepared solution of 1CNC/4ChNF/PVA at 95% water. This sample was then placed in the freezer and subjected to up to five freeze-thaw cycles. This number of FT cycles was chosen given its similarity to the measured mechanical properties of natural NPs.

The resulting 10 mm tall and 56 mm wide hydrogel/aerogel hybrid sample was compressed between plates in order to mechanically characterize it and compare to the average human IVD. After compression, the plates were then reversed at the same strain rate in order to generate hysteresis curves and demonstrate the recovery behavior of the material. Compression was performed at a strain rate of 2 mm per minute to 50% compression.

The tested sample exhibited behavior that was similar to that of natural IVDs with mechanical properties summarized in the table below. The peak stress achieved at 50% compression was approximately 690 kPa, which was within the reasonable range of pressure expected for an IVD. The stress value for this hybrid material was nearly 10× larger than the highest stress achieved by the 1CNC/4ChNF hydrogel (71.5 kPa), indicating a high level of support provided by the aerogel construct. The shape of the stress-strain curve was more similar to those of the hydrogel samples did not possess the three-stage compression shape of the stress-strain curves associated with an aerogel.

It was possible that the introduction of the hydrogel may have led to increased flexibility of the aerogel struts, resulting in a more hydrogel-like mechanical response. The elastic modulus of this hybrid material after the first 5% of compression was 94 kPa, which was within the range reported for the larger lumbar discs' annulus fibrosus (75.8-110.7 kPa). Additionally, the energy associated with deformation of the material upon loading and unloading was 100 kPa and 24.1 kPa, respectively, indicating a total loss of approximately 76 kPa of energy. This energy loss was attributed to some plastic deformation of the hybrid material, which could be investigated further in fatigue testing for long-term performance.

Mechanical property summary of IVDs. Hydrogel/Aerogel hybrid material highlighted in grey for comparison.

| | NP or AF | Men or Women | Spinal Region | Modulus (MPa) |
|---|---|---|---|---|
| Invention | Both | | Lumbar | 0.094 |
| Prior art | AF | NA | C3-C6 | 1.0-4.2 |
| | Both | M | Thoracic | 9.7-17.7 |
| | Both | W | Thoracic | 11.5-21.3 |
| | NP | NA | L3-L5 | 0.0058 |
| | AF | NA | L3-L5 | 0.0758-0.1107 |

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. A hybrid material, comprising polyvinyl alcohol and filler comprising nanocrystalline cellulose and chitin nanofibers.

2. The hybrid material according to claim 1, wherein the polyvinyl alcohol has a weight average molecular weight from 50,000-250,000 Da.

3. The hybrid material according to claim 1, wherein the polyvinyl alcohol has a weight average molecular weight from 146,000-186,000 Da.

4. The hybrid material according to claim 1, wherein the nanocrystalline cellulose has a first dimension that is 50-250 nm, and a second dimension that is 1-10 nm.

5. The hybrid material according to claim 1, wherein the chitin nanofibers have a first dimension that is 250-1,000 nm, and a second dimension that is 1-10 nm.

6. The hybrid material according to claim 1, wherein the filler is present in an amount from 1-10 wt. % relative to the total weight of the hybrid material.

7. The hybrid material according to claim 1, wherein the filler comprises nanocrystalline cellulose and chitin nanofibers in a weight ratio of 1:1 to 1:5.

8. The hybrid material according to claim 1, wherein the polyvinyl alcohol is present in an amount of 94 wt. %, the crystalline nanocellulose is present in an amount of 1 wt. %, and the chitin nanofibers are present in an amount of 4 wt. %.

9. The hybrid material according to claim 1, further comprising water in an amount of at least 90 wt. %, relative to the total weight of the hybrid material.

10. The hybrid material according to claim 1, comprising no more than 5 wt. % of a solvent, relative to the total weight of the hybrid material.

11. A method of making a hybrid material hydrogel, comprising:
a) providing a first mixture of polyvinyl alcohol in water,
b) adding chitin nanofibers to the first mixture to provide a second mixture,
c) adding nanocrystalline cellulose to the second mixture to provide a third mixture, and
d) subjecting the third mixture to one or more freeze-thaw cycles to provide a hybrid material hydrogel.

12. The method according to claim 11, further comprising lyophilizing the hybrid material subsequent to the freeze-thaw cycles to provide an aerogel.

13. The method according to claim 11, wherein the first mixture further comprises an acid.

14. A hybrid material, prepared by the process according to claim 11.

15. An implant, comprising:
a) an inner portion comprising a hydrogel comprising water, polyvinyl alcohol, and a filler, said filler comprising nanocrystalline cellulose and chitin nanofibers, said hydrogel comprising water in an amount of at least 90 wt. %, relative to the total weight of the hydrogel; and
b) an outer portion encircling the inner portion, said outer portion comprising an aerogel comprising polyvinyl alcohol and a filler comprising chitin nanofibers, said aerogel having no more than 1 wt. % solvent, relative to the total weight of the aerogel.

16. The implant according to claim 15, wherein the filler in the hydrogel comprises nanocrystalline cellulose and chitin nanofibers in a weight ratio of 1:1 to 1:5.

17. The implant according to claim 15, wherein the filler in the aerogel consists of chitin nanofibers.

18. The implant according to claim 15, wherein the aerogel consists of polyvinyl alcohol and chitin nanofibers in a 99:1 weight ratio.

19. The implant according to claim 15, wherein the hydrogel in the inner portion is prepared by a process comprising the steps:
a) providing a first mixture of polyvinyl alcohol in water,
b) adding chitin nanofibers to the first mixture to provide a second mixture,
c) adding nanocrystalline cellulose to the second mixture to provide a third mixture, and
d) then subjecting the third mixture to one or more freeze-thaw cycles to provide the hydrogel.

20. The implant according to claim 15, wherein the aerogel in the outer portion is prepared by a process comprising the steps:
a) providing a first mixture of polyvinyl alcohol in water,
b) adding chitin nanofibers to the first mixture to provide a second mixture,
c) subjecting the second mixture to one or more freeze-thaw cycles to provide a hydrogel, and
d) lyophilizing the hydrogel to provide an aerogel.
